(19) United States
(12) Patent Application Publication
(10) Pub. No.: US 2017/0027479 A1
(43) Pub. Date: Feb. 2, 2017

(54) Systems and Methods for Rehabilitating the Hand

(71) Applicant: Flint Rehabilitation Devices, LLC, Irvine, CA (US)

(72) Inventors: Nizan Friedman, Irvine, CA (US); Daniel K. Zondervan, Long Beach, CA (US); David J. Reinkensmayer, Irvine, CA (US); Mark Bachman, Irvine, CA (US)

(21) Appl. No.: 15/303,402

(22) PCT Filed: Apr. 17, 2015
(86) PCT No.: PCT/US15/26365

§ 371 Date: Oct. 11, 2016

Related U.S. Application Data
(60) Provisional application No. 61/980,680, filed on Apr. 17, 2014.

(51) Int. Cl. A61B 5/11; A63F 13/24; A63F 13/42; A63F 13/212

(52) U.S. Cl. CPC A61B 5/1125; A63F 13/212; A63F 13/24; A63F 13/42; A61B 2505/09

(57) ABSTRACT

In some embodiments, a user interface device includes a body adapted to overlie an outer side of a hand of a user when the device is worn by the user, elongated finger straps extending from a distal edge of the body, an elongated thumb strap extending from a side edge of the body, and a finger cot attached to a distal tip of each finger strap and the thumb strap, each finger cot being adapted to fit over a fingertip or thumb tip of the user, each finger cot comprising a discrete conductive pad that covers only a portion of the finger cot, wherein contact between the conductive pad of the finger cot of the thumb strap and the conductive pad of the finger cot of a finger strap can be detected.

### CROSS-REFERENCE TO RELATED APPLICATION

This application claims priority to co-pending U.S. Provisional Application Ser. No. 61/980,680, filed Apr. 17, 2014, which is hereby incorporated by reference herein in its entirety.

### BACKGROUND

Stroke remains the leading cause of chronic adult disability in western countries with over four million survivors currently living in the United States. Following the onset of stroke, patients may receive several weeks of intensive rehabilitation in an attempt to increase cognitive and functional abilities. Through intensive and repetitive motion training, patients may be able to regain lost function through processes such as neural reorganization.

Unfortunately, the length of stay at in-patient rehabilitation facilities may be limited to a few weeks and follow-up outpatient therapy is also often limited as well. Accordingly, patients must independently continue their therapy at home without access to specialized equipment or professional supervision. For example, an important task for patients with hand impairment is to repeatedly practice making coordinated finger and thumb movements that humans use to manipulate objects, such as the pincer grip, the key-pinch grip, and finger-thumb opposition. Rehabilitation therapists and scientists believe such movements are important for patients to practice because they are the movements that patients must master to be able to use the hand to manipulate objects in daily life. Further, motor learning research has shown that motor learning does not transfer well to other tasks besides the ones practiced. In addition, motor learning and rehabilitation science has shown that patients must practice such movements thousands of times to reach the skill level that they have the potential to achieve.

Unfortunately, without the presence of a clinician, practicing such movements is neither engaging nor does it provide any quantitative measure of improvement. As a result, patients often lose motivation to perform independent rehabilitation. Without ongoing practice in using the hand, individuals can experience declines in motor function, including a decrease in hand motor ability that affects their ability to perform activities of daily living. What is needed and is not currently available is a system and method that enables patients with hand impairment to independently practice the gripping movements required to manipulate objects in a motivating environment that provides quantitative feedback.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a top view of an embodiment of a user interface device that can be worn by a user.

FIG. 2 is a bottom view of the user interface device of FIG. 1.

FIG. 3 is a top view of the user interface device of FIG. 1 that shows the internal components of the device.

FIG. 4 is a top view of the user interface device of FIG. 1 as worn by a user.

FIG. 5 is a bottom view of the user interface device of FIG. 1 as worn by a user.

FIG. 6 is a side view of the user interface device of FIG. 1 as worn by a user.

FIG. 7 is a first use example illustrating a user touching his thumb to the tip of his middle finger.

FIG. 8 is a second use example illustrating a user forming a pincer grip.

FIG. 9 is a third use example illustrating a user forming a key pinch grip.

FIG. 10 is a schematic view of an embodiment of a hand rehabilitation system that includes a user interface device and a computer.

FIG. 11 is an image of a menu screen of a graphical user interface (GUI) of an embodiment of a hand rehabilitation program.

FIG. 12 is an image of a game screen of the hand rehabilitation program GUI.

FIG. 13 is an image of a results screen of the hand rehabilitation program GUI.

FIG. 14 is an image of an analytics screen of the hand rehabilitation program GUI.

FIG. 15 is a block diagram of an example configuration of a computer that executes a hand rehabilitation program.

### DETAILED DESCRIPTION

As described above, needed is a system and method that enables patients with hand impairment to independently practice the gripping movements required to manipulate objects in a motivating environment that provides quantitative feedback. Disclosed herein are examples of such systems and methods. In one embodiment, a system comprises a user interface device that is worn on the hand that can detect when the wearer successfully performs particular hand grips. The user interface device can comprise a minimalistic “glove” that includes finger cots that fit over the tips of the fingers and thumb. Conductive pads are provided on the finger cots and, when the conductive pad of the thumb is placed in contact with a conductive pad of a finger, the contact can be detected. In some embodiments, the user interface device can further comprise a finger loop that includes a conductive pad that can be worn on one of the user's fingers, such as the index finger. In such cases, contact between the tip of the thumb and the side of the finger can also be detected. In some embodiments, the user interface device is used in conjunction with a hand rehabilitation program that operates on a computer. The program includes a graphical user interface (GUI) that provides visual cues as to which hand grip the user is to form and when. The accuracy of the formation of the grips, including the timing of their formation, can be identified by the program and communicated to the user to provide feedback to the user. In some embodiments, the program is a game that reduces the tedium ordinarily associated with forming the various grips.

In the following disclosure, various specific embodiments are described. It is to be understood that those embodiments are example implementations of the disclosed inventions and that alternative embodiments are possible. All such embodiments are intended to fall within the scope of this disclosure.

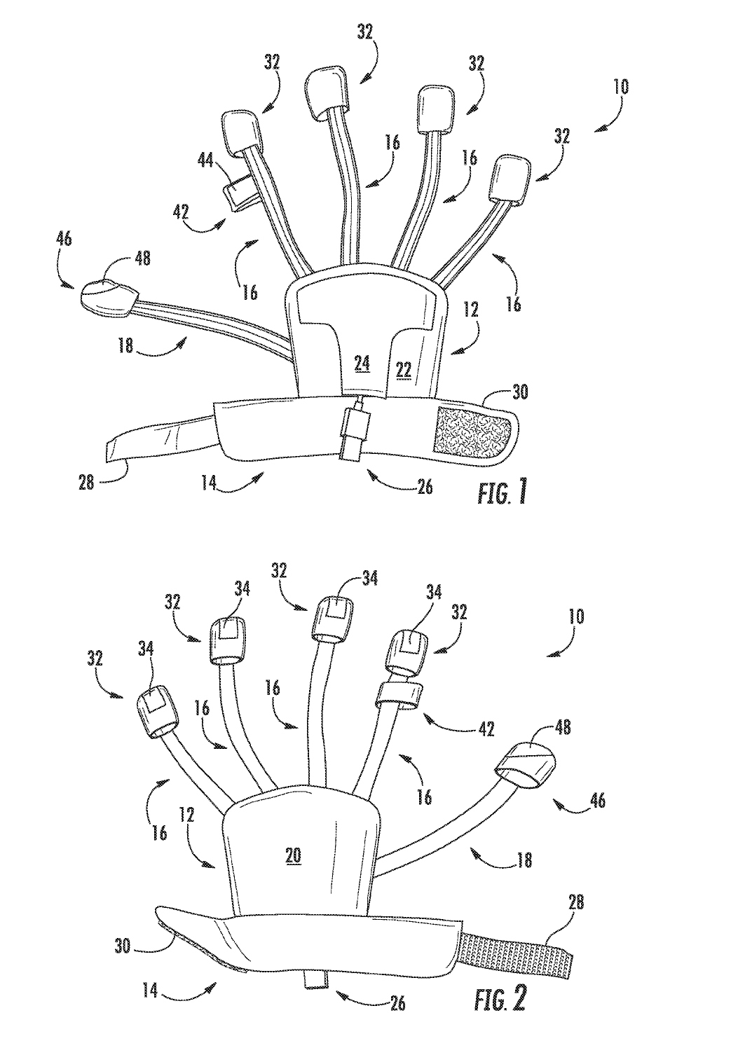

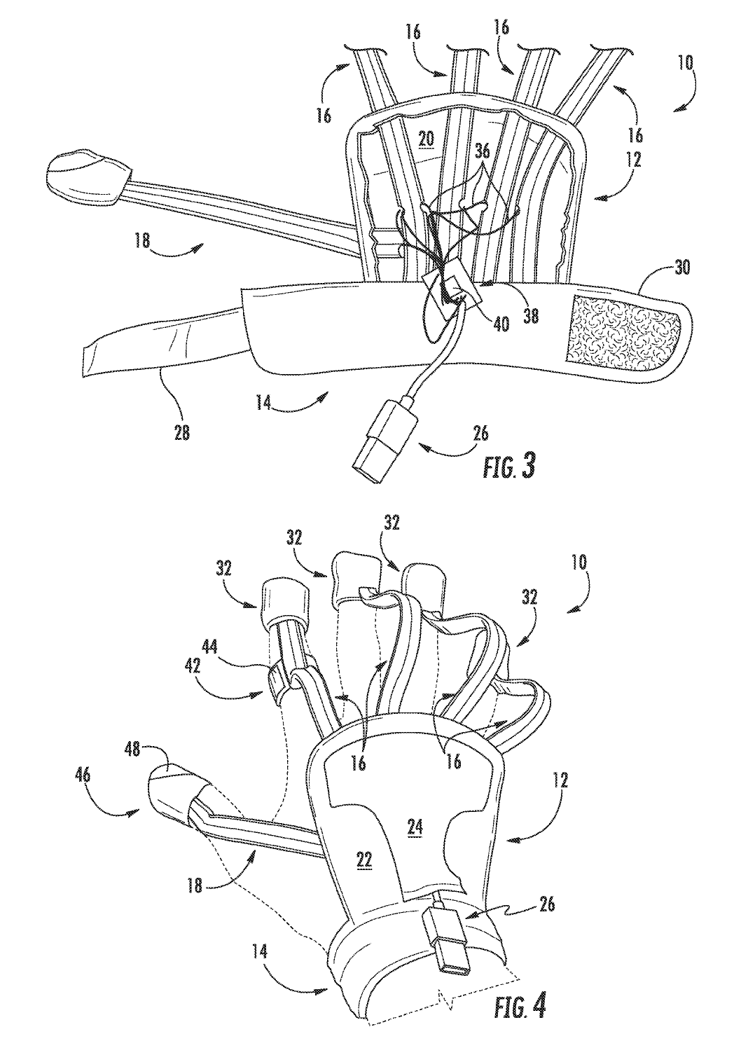

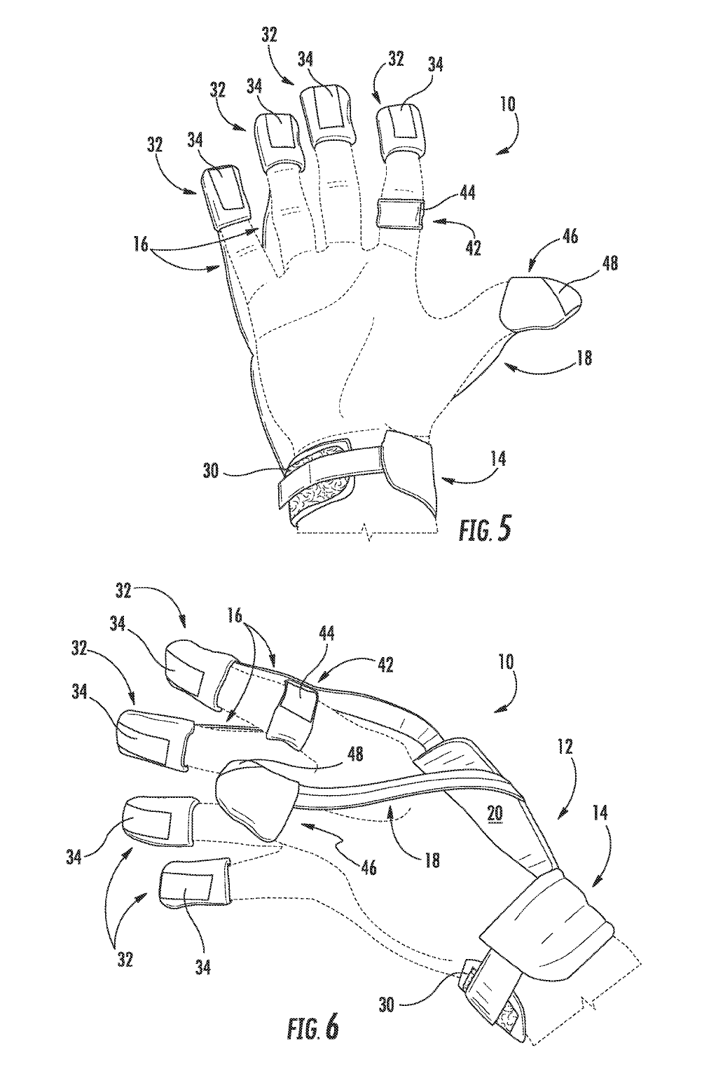

FIGS. 1 and 2 illustrate a user interface device 10 that can be worn on a hand for which rehabilitation is needed. As shown in FIG. 1, the device 10 generally comprises a body 12 to which is connected a wrist strap 14, multiple finger straps 16, and a thumb strap 18, which may also be referred to as a “finger” strap. The body 12 comprises a generally planar and rectangular member that is composed of multiple layers of material. In the illustrated embodiment, these layers of material include an inner layer 20, an intermediate layer 22, and an outer layer 24. By way of example, each of these layers 20-24 can be made of an elastic material, such as neoprene or spandex fabric. In such a case, the various layers can be sewn together to form the structure of the body 12. Irrespective of its construction, the body 12 is adapted to overlie the outer side of the hand when the device 10 is worn (see FIG. 4). As shown in FIG. 1, an electrical cable 26 extends out from a space formed between the intermediate layer 22 and the outer layer 24. As described below, this cable 26 can be used to transfer data sensed by the device 10 to another device, such as a computer.

The wrist strap 14 is attached to the body 12 along its proximal edge. As shown in the figures, the wrist strap 14 is elongated and extends laterally outward from the sides of the body 12 so that it is adapted to wrap around the wrist of the user. As shown in the figures, a first fastening element 28 is provided at one end of the wrist strap 14 and a second, complementary fastening element 30 is provided at the other end of the wrist strap. By way of example, one of the fastening elements comprises a piece of hook material and the other fastening element comprises a piece of loop material so as to together form a hook-and-loop fastener.

With further reference to FIGS. 1 and 2, the finger straps 16 are narrow, elongated members that extend from a distal edge of the body 12. Like the body 12, the finger straps 16 can be made of an elastic material, such as neoprene or spandex fabric. In some embodiments, the finger straps 16 are made from two pieces of neoprene fabric that are sewn together in a manner in which one or more internal passages are formed through which one or more conductive wires can pass.

Provided at the distal end of each finger strap 16 is a finger cot 32 that comprises an opening in which the user can place a fingertip (see FIGS. 4-6). In some embodiments, each finger cot 32 is elastic so that the finger cot snuggly grips the fingertip on which it has been placed. In such a case, the finger cot 32 can be positioned in a desired orientation on the finger and will remain in that orientation until intentionally changed by the user. When the finger cots 32 have such elasticity, they can be made of a non-conductive elastic material, such as such as neoprene or spandex fabric. In some embodiments, the finger cots 32 are also lined with an inner antimicrobial, antifungal medical fabric (not visible) that discourages the growth of bacteria and fungi.

As is shown most clearly in FIG. 2, a conductive pad 34 is provided on a portion of each finger cot 32. More particularly, the conductive pads are provided on the inner side of each finger cot 32 near its tip, which corresponds to the pad of the user's finger when the user interface device 10 is worn. In some embodiments, each conductive pad 34 comprises a discrete rectangular patch of conductive material. By way of example, the conductive material can be a metallized fabric, such as nylon that has been plated with a conductive metal such as copper, silver, or nickel. Regardless of its construction, each conductive pad 34 only covers a small fraction of the outer surface area of the figure cot 32, the remainder of which being made of a non-conductive material. Therefore, as shown in FIG. 2, the conductive pads 34 do not extend beyond the inner side of the tip of its associated finger cot 32. Each conductive pad 34 is connected (e.g., soldered) to an internal conductive wire that extends from the pad, along the internal passage of the associated finger strap 16, and to a microcontroller housed within the body 12. FIG. 3 shows an example of such an arrangement. In FIG. 3, the intermediate and outer layers 22, 24 have been removed to reveal an interior space of the body 12. As is apparent from FIG. 3, the finger straps 16 can extend through the body 12 and secure to the wrist strap 14 to improve the durability of the user interface device 10. In addition, a conductive wire 36 can extend from the internal passage of each finger strap 16 and connect to a small printed circuit board (PCB) 38 on which a microcontroller 40 is mounted. Also connected to the PCB 38 is the electrical cable 26 shown in FIG. 1. In some embodiments, the electrical cable 26 comprises a universal serial bus (USB) cable.

With reference back to FIGS. 1 and 2, one of the finger straps 16 (e.g., the finger strap associated with the user's index finger) includes a finger loop 42 through which a finger (e.g., the index finger) can be passed. The finger loop 42 can be secured to the finger strap 16 so that it cannot move along the length of the strap. Like the finger cots 32, the finger loop 42 can be made of a non-conductive elastic material and comprises a conductive pad 44, which can take the form of a rectangular patch of conductive material, such as a metallized fabric. As is apparent from FIG. 6, the conductive pad 44 is positioned on the finger loop 42 so that, when the user interface device 10 is worn on the hand, the conductive pad is positioned on the side of the finger that faces the thumb. Like the other conductive pads 34, the conductive pad 44 is connected (e.g., soldered) to an internal conductive wire that extends from the pad, along the internal passage of the associated finger strap 16, and to the microcontroller 40 housed within the body 12 (see FIG. 3).

As shown in FIGS. 1 and 2, the thumb strap 18 extends from a side edge of the body 12. The thumb strap 18 has a design and construction similar to that of the finger straps 16. Accordingly, the thumb strap 18 can be a narrow, elongated member that is made of an elastic material, such as neoprene or spandex fabric, and can include one or more internal passages through which a conductive wire can be passed. Provided at the distal end of the thumb strap 18 is a further “finger” cot 46 that comprises an opening in which the user can place the thumb tip (see FIGS. 4-6). In some embodiments, the finger cot 46 is made of an non-conductive elastic material, such as such as neoprene or spandex fabric, and is lined with an inner antimicrobial, antifungal medical fabric (not visible) that discourages the growth of bacteria and fungi.

As shown most clearly in FIG. 2, a conductive pad 48 is provided at the tip of the finger cot 46. More particularly, the conductive pad 48 is provided on an inner side of the tip of the finger cot 46 that corresponds to the user's thumb pad and faces the index finger when the user interface device 10 is worn (see FIG. 4). This conductive pad 48 can also comprise a rectangular patch of metallized fabric and can be connected (e.g., soldered) to an internal conductive wire that extends from the pad, along the internal passage of the thumb strap 18, and to the microcontroller 40 housed within the body 12 (FIG. 3).

FIGS. 4-6 illustrate the user interface device 10 as worn on the hand of a user. As shown in these figures, the body 12 can be placed on top of the outer side (back) of the hand, the wrist strap 14 can be secured around the wrist, each of the fingertips can be placed within a finger cot 32, and the thumb tip can be placed within the finger cot 46. As is also shown in these figures, the index finger has been passed through the finger loop 42. When the device 10 is worn in this manner, the user can move the hand and fingers throughout their full range of movement, whatever that may be at the time. This capability can be, at least in part, facilitated by the elastic nature of various components of the device 10 as well as the fact that the device excludes much of the material that is provided in a conventional glove. The device 10, therefore, does not impede movement of the hand or fingers, or the formation of various hand grips, which may require the touching of the thumb tip to a fingertip or a side of a finger.

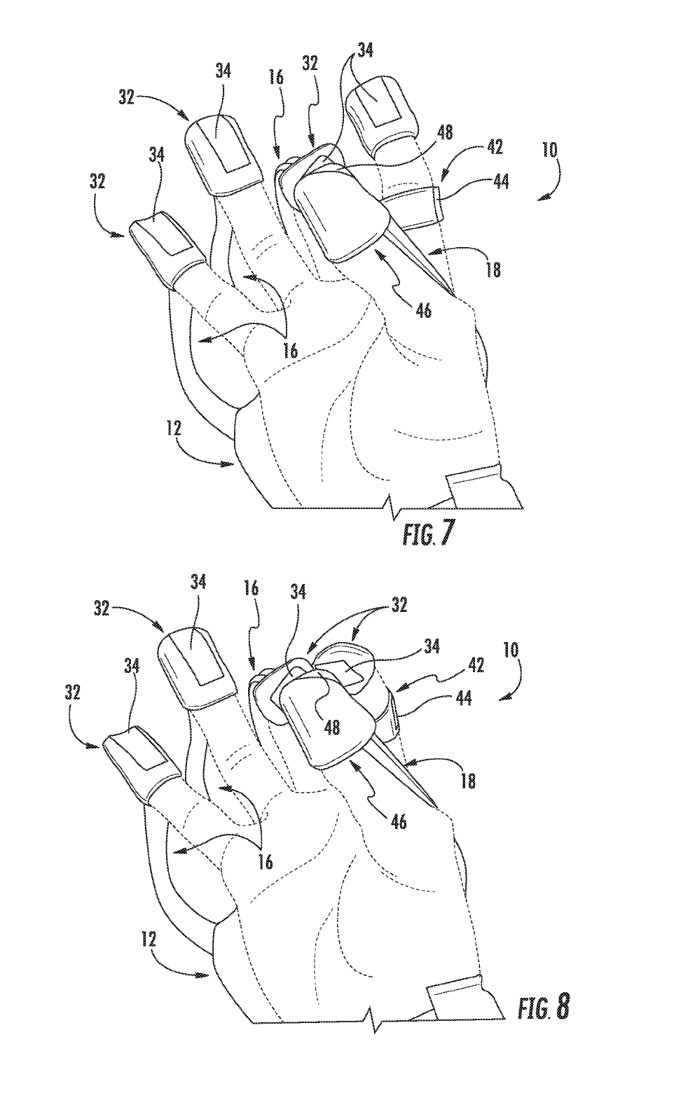

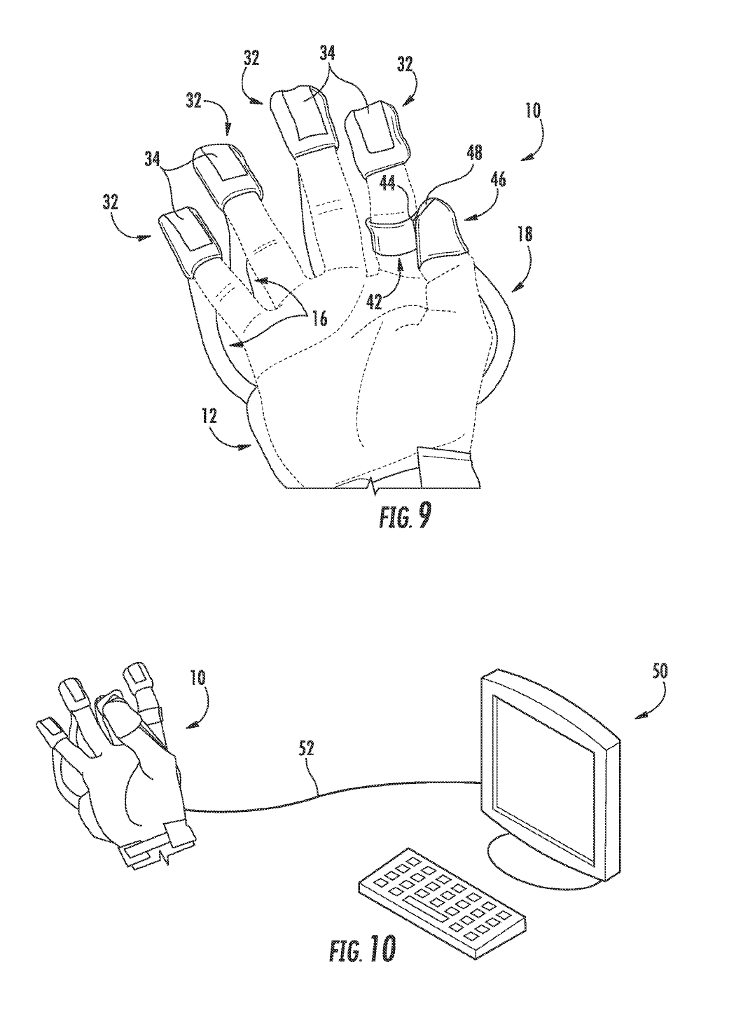

FIGS. 7-9 illustrate example hand grips that can be formed by the user while wearing the user interface device 10. Beginning with FIG. 7, the user has touched his thumb tip to the tip of his middle finger to form a pincer grip. In such a case, contact is made between the conductive pad 48 provided on the finger cot 46 worn on the thumb and the conductive pad 34 provided on the finger cot 32 worn on the middle finger. When powered by the electrical cable 26, the microcontroller 40 can detect this contact, the time at which it occurred, and the duration for which it occurred. By way of example, a voltage can be applied to the conductive pad 34 of a finger cot 32 and contact between that conductive pad and the conductive pad 48 of the thumb's finger cot 46 completes a circuit, and this occurrence can be detected by the microcontroller. Turning to FIG. 8, the user has touched his thumb tip to the tips of his index and middle fingers to form a pinch grip. In such a case, contact is made between the conductive pad 48 provided on the finger cot 46 worn on the thumb and the conductive pads 34 provided on the finger cots 32 worn on the index and middle fingers. Significantly, because the conductive pads 34 cover only discrete portions of the finger cots 32 and because the finger cots are made of a non-conductive material, contact between the conductive pad 48 and either conductive pad 34 will only be detected when actual physical contact is made between the thumb tip and the fingertip. Referring next to FIG. 9, the user has touched his thumb tip to the side of his index finger to form a key pinch grip. In such a case, contact is made between the conductive pad 48 provided on the finger cot 46 worn on the thumb and the conductive pad 44 provided on the finger loop 42 worn on the index finger.

Rehabilitation exercises can be performed using the user interface device 10. More particularly, the device 10 can be used to perform various hand grips and the device 10 can be used to confirm that the grips were correctly performed by detecting contact between the thumb and a finger of the hand. In some embodiments, the rehabilitation exercises can be performed while utilizing a hand rehabilitation program that operates on a computer to which the device 10 is connected via the electrical cable 26. This is illustrated in FIG. 10, which shows the device 10 coupled to a computer 50 with the cable 52. For example, a program that executes on the computer 50 and instructs the user in a game setting to form particular hand grips at particular points in time can be used to guide the user through the rehabilitation exercises and evaluate the accuracy with which they are performed. In some embodiments, the user's progress can be tracked over time to determine whether or not the user's hand motor skills are improving.

FIGS. 11-14 are images of example screens of a GUI of an example hand rehabilitation program of the type described above. FIG. 11 shows a menu screen 60 that can be used to make various selections for the exercise session, including the number of hand grip types that will be practiced during the session (e.g., 3, 4, or 5 types), the level of difficulty of the session (e.g., easy, medium, or hard), and the duration of the session (e.g., 15 min., 30 min., 45 min., or 60 min.). In addition, the various hand grips can be illustrated on the menu screen 60 and the particular grips that the user will be called upon to practice can be highlighted based upon the number of grip types the user selects.

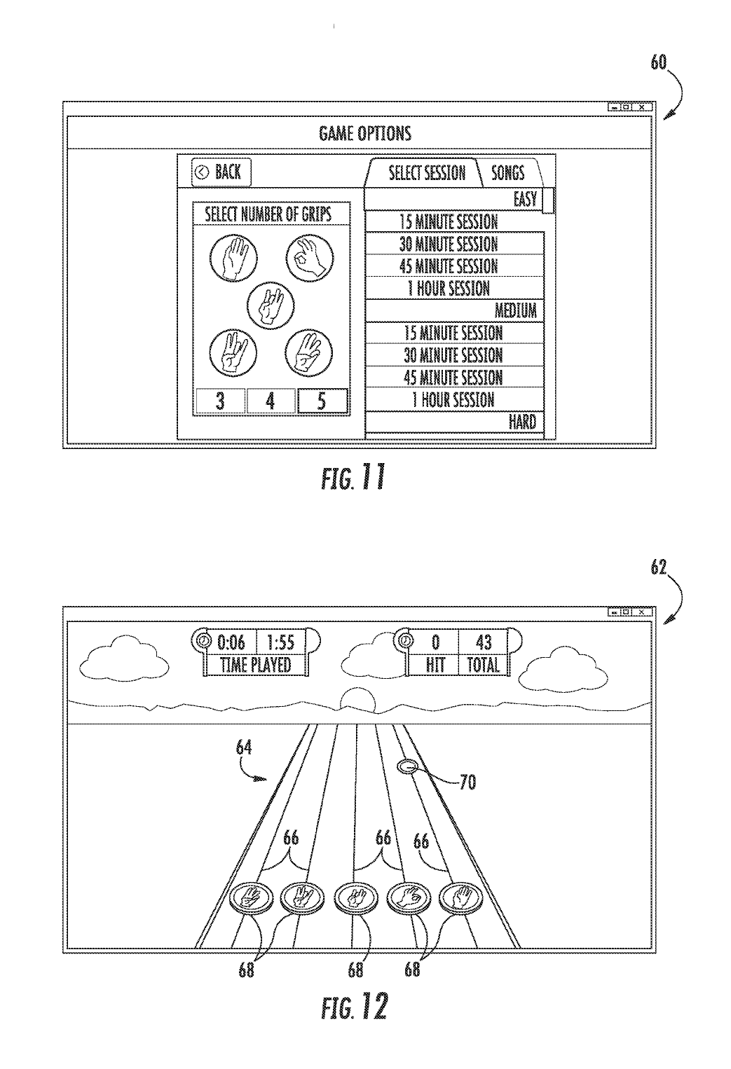

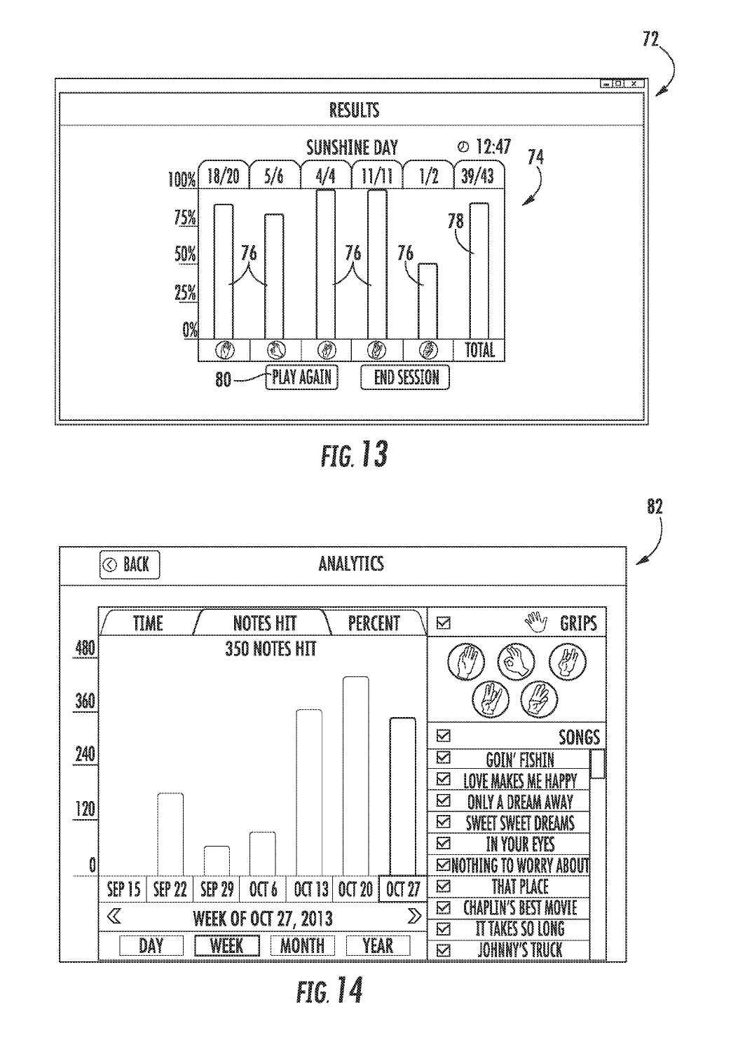

Once the user selections have been made, the exercise session can begin. In some embodiments, a game screen 62 shown in FIG. 12 is presented to the user. In this game screen 62, a fret board 64, such as that typically found on a guitar or similar instrument, is illustrated that has five strings 66, each relating to a particular hand grip that is illustrated in a circle 68 at the bottom end of each string. Ellipsoids 70 can travel down the strings 66 with a particular sequence, frequency, and speed that relate to the level of difficulty that has been selected by the user. When an ellipsoid 70 reaches the bottom of its string 66 and overlies the circle 68 provided there, the user can form the grip illustrated in the circle. If the user successfully forms the grip when the ellipsoid 70 overlies the circle 68, this success is recorded by the program. If the user fails to form the grip when the ellipsoid overlies the circle, either because he or she could not form the grip or he or she formed it too early or too late, this failure is recorded by the program. In some embodiments, songs are played during the session and the quality of the song that the user hears corresponds to the skill of the user in forming the required grips at the required times. This transforms the exercise session to a game that challenges the user and provides motivation to continue his or her rehabilitation therapy.

Referring next to FIG. 13, after a song has ended, the results of the user's grip forming can be presented to the user with a results screen 72. In the illustrated embodiment, the results screen 72 comprises a bar graph 74 that includes a separate bar 76 for each hand grip type that the user was required to form during the song as well as a bar 78 that provides an overall indication of the user's grip-making ability. As to each hand grip type, the user's ability to form the grip at the correct time is evaluated as a percentage, with 100% indicating that the each grip was properly formed at the right time each time the program indicated that it should be formed. The game may also display the score of previous attempts at the song. When the user selects the “Play Again” button 80, the next song can be played for the user and the results of that song can be presented. Operation continues in this manner until the user has exercised for the full duration that was selected on the menu screen 60. Once the session is over, results for the entire session can be presented to the user. In addition, this information can be shared with the user's physician and/or physical therapist.

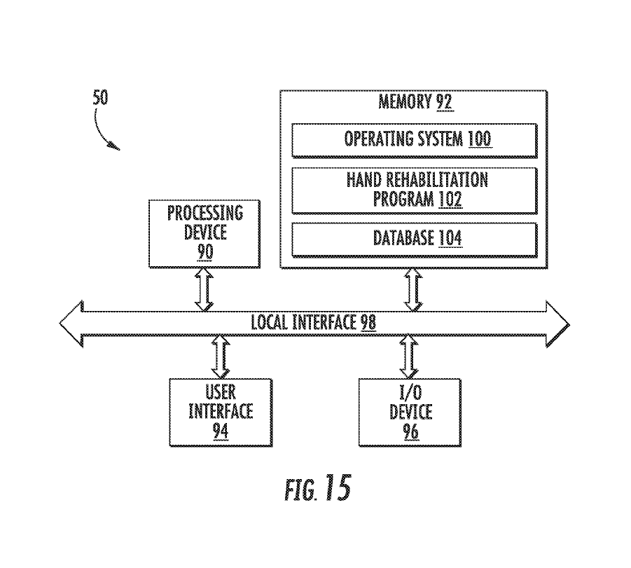

In some embodiments, the GUI can further include an analytics screen 82 shown in FIG. 14. This screen 82 enables users to track their progress over time. In some embodiments, the analytics screen enables users to track total time played, number of notes hit, or percentage of correct/total notes hit; bins results in day, week, month, and year intervals; bins results by types of grips (one can chose any number of grips); and bins results by songs (one can chose any number of songs).

It is further noted that the chosen metrics in the game are highly correlated with established clinical assessments of hand function. One such clinical assessment is called the “box and block” test.

FIG. 15 is a block diagram of an embodiment for the computer 50 that can be used in the hand rehabilitation system. As shown in this figure, the computer 50 generally comprises a processing device 90, memory 92, a user interface 94, and an input/output (I/O) device 96, each of which is connected to a system bus 98.

The processing device 90 can, for example, include a central processing unit (CPU) that is capable of executing instructions stored within the memory 92. The memory 92 includes any one of or a combination of volatile memory elements (e.g., RAM) and nonvolatile memory elements (e.g., hard disk, ROM, etc.).

The user interface 94 comprises one or more devices with which a user interfaces with the computer 50. The user interface 94 can, for example, comprise a keyboard, mouse, and display. The I/O device 96 comprises a component that enables the computer 50 to communicate with other devices, such as the device 10.

The memory 92 (a non-transitory computer-readable medium) stores programs (i.e., logic) including an operating system 100 and a hand rehabilitation program 102. The operating system 100 controls the general operation of the computer 50, while the hand rehabilitation program 102 comprises the software that the user uses in conjunction with the device 10 in performing rehabilitation exercises. Also included in memory is a database 104, in which the results from the exercise sessions can be stored.

Various changes can be made to the disclosed systems and methods. For example, in some embodiments, the user interface device can include a finger loop on a finger other than the index finger. Alternatively, multiple finger loops can be used for multiple fingers. In other embodiments, a conductive pad can be provided on the outer surface of one of the finger straps (e.g., the finger strap associated with the middle finger) so that contact will be made between the conductive pad and the conductive pad of the thumb when a power grip is formed. In still further embodiments, the rehabilitation therapy can comprise adjusting the position of one or more conductive pads on the fingers or thumb by adjusting the position of its associated finger cot or finger loop to change the nature of the grip that can be detected. As noted above, when the finger cots/loops are elastic, they will maintain the position on the finger/thumb at which they are placed by the user.

Claimed are:

1. A user interface device comprising:
a body adapted to overlie an outer side of a hand of a user when the device is worn by the user;
elongated finger straps extending from a distal edge of the body;
an elongated thumb strap extending from a side edge of the body; and
a finger cot attached to a distal tip of each finger strap and the thumb strap, each finger cot being adapted to fit over a fingertip or thumb tip of the user, each finger cot comprising a discrete conductive pad that covers only a portion of the finger cot, wherein contact between the conductive pad of the finger cot of the thumb strap and the conductive pad of the finger cot of a finger strap can be detected.

2. The device of claim 1, wherein the body only overlies the outer side of the hand and no material covers the sides or the palm of the hand.

3. The device of claim 1, wherein the body is made of an elastic material.

4. The device of claim 1, wherein no material covers the user's fingers and thumb except for the finger straps and thumb strap such that the user's fingers and thumb are generally exposed.

5. The device of claim 1, wherein the finger straps and thumb strap are made of an elastic material.

6. The device of claim 1, wherein the finger cots are made of an elastic material.

7. The device of claim 1, wherein the conductive pads are provided on inner sides of the finger cots so as to overlap pads of the user's fingers and thumb when the device is worn.

8. The device of claim 1, wherein the conductive pads comprise metallized fabric.

9. The device of claim 1, further comprising a wrist strap connected to a proximal edge of the body.

10. The device of claim 1, further comprising conductive wires that extend from the conductive pads and along an associated finger strap or thumb strap to the body.

11. The device of claim 10, further comprising a microcontroller provided in the body to which the conductive wires connect.

12. The device of claim 11, further comprising an electrical cable connected to the microcontroller that can electrically connect the device to a computer.

13. The device of claim 1, further comprising a finger loop connected to one of the finger straps wherein contact between the conductive pad of the finger cot of the thumb strap and the conductive pad of the finger loop can be detected.

14. A hand rehabilitation system comprising:
a user interface device including a body, elongated finger straps and a thumb strap extending from edges of the body, and a finger cot attached to a distal tip of each finger strap and the thumb strap, each finger cot being adapted to fit over a finger or thumb tip of a user, each finger cot comprising a discrete conductive pad that covers only a portion of the finger cot; and
a hand rehabilitation program configured to execute on a computer, wherein the user interface device can be electrically coupled to the computer and the program can identify when contact is made between the conductive pad of the finger cot of the thumb strap and the conductive pad of a finger cot of a finger strap.

15. The system of claim 14, wherein the hand rehabilitation program is configured to instruct the user to perform particular hand grips at particular times and to monitor the accuracy with which the user forms the grips.

16. The system of claim 15, wherein the hand rehabilitation program is configured as a game that the user can play.

17. The system of claim 15, wherein the hand rehabilitation program is configured to provide quantitative feedback to the user that indicates his or her accuracy in forming the particular hand grips at the particular times.

18. A method for providing hand rehabilitation to a user, the method comprising:
providing a user interface device that can be worn on the user's hand, the device including finger cots that can be worn on the tips of the user's fingers and thumb, each finger cot including a discrete conductive pad;
executing a hand rehabilitation program that instructs the user to perform particular hand grips at particular times;
detecting formation of the hand grips by detecting contact between the conductive pad of a first finger cot and a conductive pad of a second finger cot; and
determining the accuracy with which the user forms the hand grips at the particular times.

19. The method of claim 18, wherein the hand rehabilitation program is configured as a game that the user can play.

20. The method of claim 18, wherein the hand rehabilitation program is configured to provide quantitative feedback to the user that indicates his or her accuracy in forming the particular hand grips at the particular times.
